(19) United States
(12) Patent Application Publication
(10) Pub. No.: US 2006/0281968 A1
(43) Pub. Date: Dec. 14, 2006

(54) System, including method and apparatus for percutaneous endovascular treatment of functional mitral valve insufficiency

(76) Inventors: Carlos M.G. Duran, Missoula, MT (US); Mark L. Sanz, Missoula, MT (US); Jose Luis Martinez de Ubago, Missoula, MT (US); Yon Patrik Martinez de Ubago, Missoula, MT (US)

Correspondence Address: GABOR L. SZEKERES, 8141 E. KAI SER BLVD., SUITE 112, ANAHEIM HILLS, CA, 92808, US

(21) Appl. No.: 11/447,648

(22) Filed: Jun. 6, 2006

Related U.S. Application Data
(60) Provisional application No. 60/688,319, filed on Jun. 7, 2005.

(51) Int. Cl. A61F 2/06; A61M 29/02; A61F 2/94

(52) U.S. Cl. 600/37; 623/1.15; 606/194; 623/1.21

(57) ABSTRACT

Among the four heart valves, the mitral is the most frequently affected by disease resulting in defective valve opening (stenosis) or incomplete closure (insufficiency). Most often this is due to distortion of the valve apparatus secondary to rheumatic or degenerative disease. These lesions, called “organic” require open heart surgery. In patients with coronary disease or with dilated cardiomyopathy the mitral valve can be insufficient although structurally normal. These valves are “functionally” insufficient. Because of the poor condition of these patients where open heart surgery carries a significant operative risk, less invasive percutaneous alternatives are being explored today. The present novel invention represents a radical departure from other procedures because it repositions the posterior papillary muscle utilizing a device located in the interventricular veins.

### CLAIM OF PRIORITY OF PROVISIONAL APPLICATION

The present application claims the priority of provisional application Ser. No. 60/688,319, filed on Jun. 7, 2005.

### BACKGROUND OF THE INVENTION

1. Field of the Invention

The present invention is in the novel field of percutaneous treatment of heart valve disease and in particular of the so called “functional” mitral valve insufficiency.

More specifically, the present invention relates to apparatus and methods for treating mitral valve insufficiency in cases where the mitral valve, although structurally intact, leaks because of changes in its geometry. These so-called “functional” mitral regurgitations are typically present in patients with coronary (ischemic) disease or with dilated cardiomyopathy. The present invention is a completely original departure from the prior art involving the restoration of the mitral valve papillary muscle geometry through the percutaneous placement of a device in the posterior, anterior or both interventricular veins of the heart.

2. Description of Relevant Anatomy and Nature of the Disease or Condition to which the Present Invention is Directed

### THE MITRAL VALVE

The mammalian circulation needs the presence of one-way valves to maintain forward blood flow. The mitral valve is the primary inflow valve controlling flow between the lungs and the main pumping chamber of the heart, the left ventricle. Either a leak or a narrowing of the mitral valve has dramatic consequences on the overall function of the left ventricle. The mitral valve is composed of several interrelated structures: 1) two translucent flaps or leaflets attached to a more or less fibrous ring or annulus; 2) a complex series of fibrous strands or chordae tendinae that connect the leaflets to two muscular pillars or papillary muscles that are part of the left ventricular wall. Pathologic alteration of any or all of these structures results in mitral insufficiency. Diseases such as rheumatic fever and degenerative or myxomatous lesions distort the valve elements through fibrosis, elongation or rupture. Conversely, some diseases such as coronary insufficiency, myocardial infarction and dilated cardiomyopathy induce a geometric change in the left ventricular wall that alters the delicate closing mechanism of an otherwise structurally normal mitral valve. Modem diagnostic techniques have shown that these so-called functional mitral regurgitations are very frequent and prevalent among our progressively aging population.

### THE BLOOD SUPPLY TO THE HEART

The heart muscle has a dedicated blood supply with a specific arterial and vein network. The oxygenated blood is supplied to the heart through two coronary artery openings, or ostia, arising at the aortic root which split into three main coronary arteries in the human. Branches of these supply oxygenated arterial blood to the muscle. De-oxygenated venous blood leaves the heart through small veins that drain directly into the heart cavities or through veins that follow a parallel course with the epicardial arteries. The main venous system consists of several branches that empty into a large Coronary Sinus that opens into the right atrium. The main veins that drain into the coronary sinus are the anterior and posterior interventricular veins that run parallel to the left anterior descending artery and posterior interventricular artery. A marginal vein that runs parallel to the marginal artery also drains into the coronary sinus. Anatomically, the coronary sinus runs parallel to part of the circumflex artery and surrounds the mitral annulus for approximately 60% of its circumference. The posterior interventricular vein arises at the ventricular apex and runs towards the base of the heart to drain into the coronary sinus very close to its termination in the right atrium. In fact, percutaneous catheterization of this vein through a femoral or jugular approach is technically very simple. This vein is fairly large with an approximate diameter of 3-5 mm. in its middle course. In relation with the present invention an important characteristic of this vein is that its epicardial course corresponds with the endocardial location of the posterior papillary muscle.

### MECHANISMS OF FUNCTIONAL MITRAL REGURGITATION

While the mechanisms responsible for organic regurgitations are very well established, the causes of functional regurgitation remain obscure. Organic lesions secondary to rheumatic fever are primarily due to fibrosis of the mitral valve complex. The leaflets become thickened, retracted and the chords are shortened. Organic lesions due to degenerative disease result in redundant tissue with enlarged leaflets, elongated chords and dilated annulus. Long-term, insufficiency causes failure of the left ventricle and changes the geometry when the failing ventricle dilates. On the other hand, functional mitral valve regurgitation secondary to coronary insufficiency, myocardial infarction, or dilated cardiomyopathy occurs in the presence of a structurally normal mitral valve. Surgical or pathologic inspection of the annulus, valve leaflets, chordae tendinae and papillary muscles is normal. However, dynamic observation particularly with echocardiography, shows significant regurgitation. The mechanisms responsible for this functional regurgitation are still debated. Initially it was thought that it was due to leaflet prolapse secondary to papillary muscle damage. Experimental models showed that papillary damage, ischemia or infarction did not induce regurgitation. Recently, an elegant echocardiographic study of patients with ischemic functional regurgitation has shown that there is no leaflet prolapse but a tenting of the leaflets towards the ventricular apex. Experimental models have confirmed that this leaflet tenting effect is due to an outward displacement of both papillary muscles and especially of the posterior papillary muscle.

### TREATMENT OF FUNCTIONAL MITRAL REGURGITATION IN ACCORDANCE WITH THE PRESENT STATE OF THE ART

Functional mitral regurgitation secondary to myocardial infarction is common with incidences between 19% and 39%. Functional mitral regurgitation has a poor prognosis with a significant difference in mortality at 5 years after infarction among patients with regurgitation (50%) versus patients without regurgitation (30%). Even mild regurgitation was associated with high mortality. In conclusion, the presence of functional mitral regurgitation after myocardial infarction caries a somber prognosis. This data demand an aggressive treatment.

The majority of patients are still treated surgically because of the lack of a simple, rapid, and minimally traumatic technique that at least would reduce the severity of the regurgitation during the acute phase of the myocardial infarction. Both acute and chronic functional mitral regurgitation are being treated surgically with coronary bypass revascularization followed by the insertion of a mitral annuloplasty ring or band. The aim of the annuloplasty is to significantly reduce the mitral annulus in order to increase leaflet apposition. Although the results have been satisfactory, the poor condition of these patients together with the need for major surgery just to place an annuloplasty device has stimulated a search for and development of simpler and less traumatic percutaneous interventions.

### DESCRIPTION OF PRIOR ART

The large number of methods known in the state-of-the-art for the percutaneous treatment of mitral regurgitation can be classified according to the approach to the mitral valve.

The first method is based on the fact that the coronary sinus surrounds part of the posterior mitral annulus. A pre-shaped band is percutaneously inserted into the coronary sinus, so that when correctly placed it cinches the mitral annulus. A representative example is described in published US patent application 2002/0016628 A1. This type of device is based on the principle that the main cause of functional regurgitation is a dilatation of the mitral annulus. These devices are limited by 1) the need for an anchoring system within the thin walled coronary sinus; 2) the anatomic fact that the coronary sinus does not surround completely the mitral annulus and 3) the percutaneous annuloplasty will be partial and not anchored on the right and left fibrous trigones crucial for the longevity of the mitral annulus contention.

A second group of devices of the state-of-the-art are based on the approximation and fixation of the mid-portion of the free edges of the anterior and posterior mitral leaflets. This technique, known as the “Alfieri stitch,” “double orifice,” or “bow-tie” because the end result is a mitral valve with two separate orifices. A representative example of these methods is described in U.S. Pat. No. 6,312,447 B1. This system requires a transeptal approach, i.e. the device that is introduced through a peripheral vein, must cross the inter-atrial septum to reach the left atrium and be placed across the mitral valve into the left ventricle. Besides the complexity of the device that must first immobilize in the closed position both anterior and posterior leaflets, a second mechanism is needed to permanently fix together the tips of the leaflets. The transeptal technique is difficult and not widely mastered by the interventional cardiologist.

A third method consists of the sectioning of the anterior mitral basal chords. Messas and associates (Messas et al., Paradoxic decrease in ischemic mitral regurgitation with papillary muscle dysfunction: insights from three-dimensional and contrast echocardiography with strain rate measurement. Circulation 2001; 104:1952-57; Messas et al., Chordal cutting: A new therapeutic approach for ischemic mitral regurgitation. Circulation 2001; 104:1958-63) have shown experimentally that section of the anterior basal chords reduces the leaflet tethering towards the apex present in functional mitral regurgitation. Basal chord sectioning increases the leaflet curvature and increases apposition. This method recently applied with open heart surgery, still awaits an endovascular technique which probably will require an arterial approach through the aortic valve.

A fourth group of devices are centered on the relocation of the papillary muscles and particularly of the posterior papillary muscle. So far, these methods require surgery although probably minimally invasive. Hung and associates have described the placement of a patch sutured to the lateral aspect of the heart incorporating a balloon that after inflation it would displace the left ventricular wall medially reducing the leaflet tenting. (Hung et al., Reverse ventricular remodeling reduces ischemic mitral regurgitation: Echo-guided device application in the beating heart. Circulation 2002; 106:2594-2600) The Coapsys (Trehan et al., Off-Pump Mitral Valve Repair Using the Coapsys™ Device: Early Results in Patients with Functional Mitral Regurgitation. Circulation 2003 Oct. 28; 108(17); 2179: IV 475. and Cardioclasp (Kashem et al., Cardioclasp changes left ventricular shape acutely in enlarged canine heart. J Cardiac Surgery 2003; Suppl 2:S49-60) devices approximate the two papillary muscles with a member that either crossing the heart or with epicardial patches held together with an external clamp mechanism can selectively bring the papillary closer together. The present invention is completely different from the above described techniques and devices.

### SUMMARY OF THE INVENTION

An original non-surgical method and apparatus for practicing the method are described for the treatment of mitral valve regurgitation. The method and apparatus are specifically suitable for treating patients having the so called “functional” mitral regurgitations where although the mitral apparatus is structurally normal the valve is incompetent because of geometric changes in the left ventricle. The novel method and apparatus utilized to implement it are percutaneous, endovascular, and completely different from all other methods previously known in the art.

The present invention is based on the following anatomical facts, observations and novel concepts.

(1) The main cause of functional mitral regurgitation is due to displacement of the papillary muscles (particularly the posterior) laterally and towards the left ventricular apex. This displacement pulls on the chordae tendinae of the mitral valve that tether down the anterior and posterior leaflets which cannot come in contact and therefore the valve becomes incompetent.

(2) The anatomic fact that the anterior and posterior interventricular veins run on the surface of the heart (epicardially) towards the left ventricular apex parallel to the endocardial papillary muscles and in particular the posterior papillary muscle. Also that these veins are not essential for the venous drainage of the heart and therefore can be occluded with impunity.

The novel concept utilized in the method and apparatus of the present invention is completely original and far simpler than other concepts, method and system of apparatus known in the previous art.

Thus, the present invention consists of a method and a system of devices designed to achieve mitral competence in cases of functional mitral regurgitation. The method of the present invention involves the endovascular medial displacement of the anterior and posterior interventricular veins towards the left ventricular cavity and therefore the medial repositioning of the papillary muscles.

The system of the present invention involves several endovascular apparatus or devices designed to be deployed within the anterior and posterior interventricular veins or only in the posterior interventricular vein. The delivery or deployment system follows the general principles well established in interventional cardiology. A percutaneous or small incision provides access to a peripheral vein (usually the femoral) and a single or double steerable guide wires are inserted through the coronary sinus opening, into the posterior or into both the posterior and anterior interventricular veins until their tips are placed close to the left ventricular apex. A single delivery catheter is then inserted following the guide wire until it is placed in the posterior interventricular vein parallel to the posterior papillary muscle. Alternatively a second guide wire is placed in the anterior interventricular vein. Guidance of the catheter/s is done under fluoroscopic control and transthoracic or transesophageal echocardiography used simultaneously to determine the degree of mitral regurgitation and location and changes in the position of the posterior papillary muscle. The delivery catheter(s) can carry a balloon, or a balloon expanding stent or a self expanding stent of a size corresponding to the size of the patient and degree of mitral regurgitation. A stiff rod, wire or plate can be incorporated into the balloon or stent to stabilize it (them) within the interventricular vein(s). A retaining endovascular plate can be also incorporated in order to limit the outward dilatation of the balloon while promoting its dilatation towards the left ventricular wall and therefore pushing medially the papillary muscle. The stent and retaining plate may be combined into another device so long as the device causes permanent medial displacement of the papillary muscle(s). Alternatively, the delivery catheter can have two small balloons placed at the apical and proximal parts of the delivery catheter so that when inflated they occlude the vein proximal and distal to the balloon or stent. Occlusion of the vein between these two points will result in clotting of the blood within these two points. This system will prevent bleeding if laceration of the vein occurs due to balloon over-dilatation. Also, the delivery catheter can have ports to administer drugs that induce blood clotting or substances that polymerize when in contact with blood between the occluding balloons.

Another aspect of the present invention is the delivery of a specifically designed eccentrically shaped, stiff, thick, and active device rod. This rod is asymmetrically shaped so as to allow for rotation of the device to put pressure against the ventricular wall. The eccentric center portion of the rod pushes against the medial portion of the vein which lies against the left ventricular wall. The rod must be able to be straightened out to go through the delivery catheter. As the catheter is pulled back, the rod remains in place and assumes spontaneously its shape. This rod is connected to a pusher wire which can be detached after the rod is properly positioned. Several methods and appropriate apparatus can be utilized to immobilize the rod once it is in the right position. The proximal and distal ends of the rod can be secured to the walls of the vein with small balloons or with mechanical devices with hooks known in the previous art. Furthermore, substances such as glues can be delivered through a catheter with multiple holes situated between the proximal and distal balloons.

In another aspect of the present invention, guide wires are placed in both the posterior and anterior interventricular veins. Small magnets are threaded through the guide wires until both veins are filled with the magnets. Their mutual attraction will bring closer both papillary muscles. Also, a similar result can be achieved by delivering through both guide wires pre- shaped, memory rods that are bound together at the level of the coronary sinus. Once the delivery catheters are removed, an inverted “U” shape device results that brings the two interventricular veins closer to one another and consequently also the papillary muscles.

The present invention is far simpler than the prior art devices and methods because (1) its percutaneous approach is standard and well known to the interventional cardiologists who have catheterized the coronary sinus for many years. (2) The entire implanted device remains in the venous system of the heart which reduces the chances of left sided thromboembolic events. (3) It allows testing of its efficacy with echo or contrast before its final implementation. (4) Possible complication of a thrombosis of the interventricular vein(s) does not carry hemodynamic consequences.

### BRIEF DESCRIPTION OF THE DRAWINGS

FIG. 1 is a schematic diagram in cross-section of the base of the heart showing the anatomical relationships of the normal mitral valve, coronary sinus and its branches such as the anterior and posterior interventricular veins and the oblique vain.

FIG. 2 is a schematic diagram in longitudinal cross section of the heart through the lateral wall of the left ventricle, showing the close anatomical relationship between the posterior papillary muscle of the mitral valve and the heart's posterior interventricular vein.

FIG. 3 is a schematic diagram of the left ventricular geometric changes leading to the appearance of “functional” mitral regurgitation with a clear arrow representing the lateral displacement of the posterior papillary muscle and a shaded arrow representing the presence of functional mitral regurgitation.

FIG. 4 is a schematic diagram of a longitudinal section of the heart where an endovascular balloon has been expanded within the posterior interventricular vein, the balloon displacing the posterior papillary muscle towards the left ventricular cavity abolishing functional mitral regurgitation, with the arrow representing medial displacement of the posterior papillary muscle.

FIG. 5 is a schematic diagram of one of the embodiments of the present invention where an endovascular stent is placed within the posterior interventricular vein, with the expanded stent causing displacement of the posterior papillary muscle towards the cavity of the left ventricle (shown by the arrow) thereby abolishing functional mitral regurgitation (shown by crossed out arrow).

FIG. 6 is a schematic diagram of the apparatus used in one of the steps in the percutaneous insertion of a balloon within the posterior interventricular vein showing a small bore catheter feeding two small balloons that occlude the vein proximally and distally to a collapsed endovascular stent.

FIG. 7 is a schematic diagram showing an alternative embodiment wherein a stiff long rod is centrally placed to reduce the lateral displacement of the balloons which are shown collapsed.

FIG. 8 is a schematic diagram showing another alternative embodiment having a central large balloon and proximal and distal hemostatic balloons (shown expanded) and a central catheter with multiple side holes designed to deliver a liquid polymer that becomes rigid at body temperature.

FIG. 9 is a schematic diagram of still another embodiment showing a pre-shaped stiff rod displacing medially the posterior interventricular vein.

FIG. 10 is a schematic diagram of a further embodiment of the apparatus and method of the present invention showing a small bore catheter having side holes through which a polymer can be injected to maintain a pre-shaped fixed rod (not shown) within appropriate position in the posterior interventricular vein.

FIG. 11 is a schematic diagram of an alternative method and apparatus to anchor a pre-shaped stiff rod to the vein by rotating an apparatus attachable to the rod (not shown) to expose several hooks to anchor the apparatus to the wall of the vein.

FIG. 12 is a schematic diagram showing the apparatus of FIG. 11 having the hooks exposed.

FIG. 13 is a schematic diagram of an alternative embodiment where both the anterior and posterior interventricular veins are used in method of the present invention first by positioning a guide wire in each vein.

FIG. 14 is a schematic diagram of a transverse section of the left ventricle at the level of the papillary muscles. Memory rods, also shown in FIG. 15, displace medially both papillary muscles.

FIG. 15 is a schematic diagram showing memory rods deployed in the anterior and posterior veins so that inverted “U” results that brings close together the veins and consequently, reduces the transverse left ventricular diameter at the level of the papillary muscles shown in transverse in FIG. 14.

FIG. 16 is a schematic diagram of a further alternative embodiment wherein segmented magnets are threaded along the anterior and posterior vein guide wires to have a magnetic attractive force to bring closer together the veins and consequently, reduce the transverse left ventricular diameter at the level of the papillary muscles.

### DESCRIPTION OF THE PREFERRED EMBODIMENTS

The following specification, taken in conjunction with the drawings, sets forth the preferred embodiments of the present invention. The embodiments of the invention disclosed herein are the best modes contemplated by the inventors for carrying out their invention in a commercial environment, although it should be understood that various modifications can be accomplished within the parameters of the present invention.

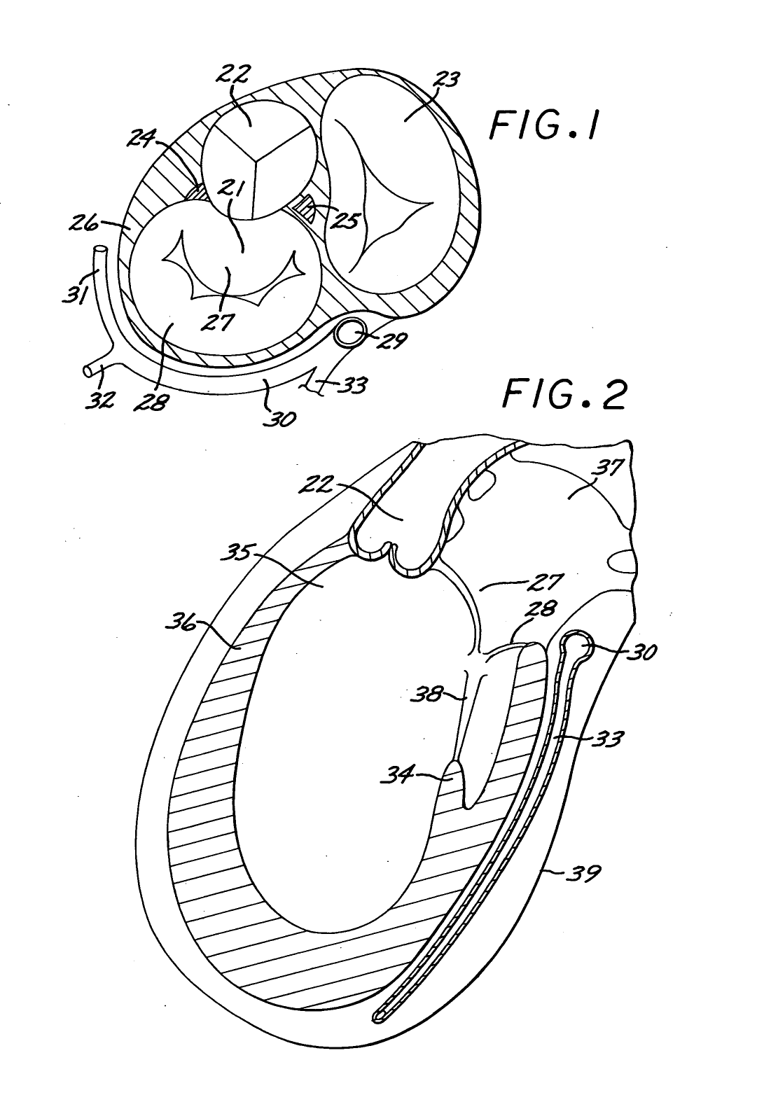

Referring now to the drawing figures, FIG. 1 is a sketch of the base of the heart which is essential for the understanding of the present invention. The mitral valve 21, aortic valve 22 and tricuspid valve 23 are shown with the left 24 and right 25 fibrous trigones of the heart supporting the mitral annulus 26 together with the anterior 27 and posterior 28 leaflets of the mitral valve 21. The coronary sinus opening into the right atrium 29 and the coronary sinus 30 with its branches are shown: the anterior interventricular vein 31, the marginal vein 32 and the posterior interventricular vein 33.

In FIG. 2 the anatomic relationship between the posterior interventricular vein 33 and the mitral valve posterior papillary muscle 34 are shown. The aortic valve 22, left ventricular cavity 35, left ventricular myocardium 36 and left atrium 37 are shown. The anterior leaflet 27 and posterior leaflet 28 of the mitral valve 21 are held by the chordae tendinae 38 attached to the posterior papillary muscle 34. The posterior interventricular vein 33 runs on the surface of the heart, from the coronary sinus 30 towards the left ventricular apex. The posterior interventricular vein 33 runs parallel to the posterior papillary muscle 34. Behind the anterior interventricular vein 31 is the pericardial membrane 39 that surrounds the heart.

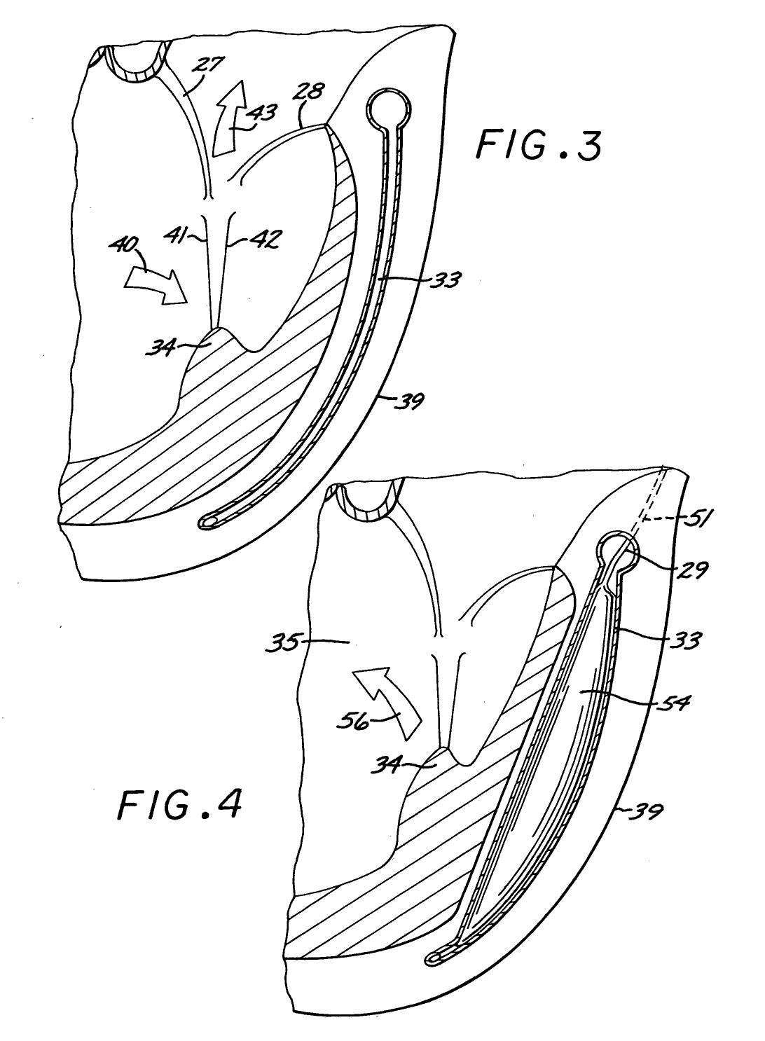

FIG. 3 is a diagrammatic description of the underlying mechanism responsible for the genesis of functional mitral regurgitation. The posterior papillary muscle 34 is displaced laterally and towards the apex of the left ventricle as shown by arrow 40. This papillary muscle displacement pulls downward the anterior 41 and posterior 42 mitral chords resulting in tethering of the anterior 27 and posterior 28 leaflets of the mitral valve 21. A functional mitral regurgitation ensues as shown by the arrow 43. The posterior interventricular vein 33 is shown running in the epicardium parallel to the posterior papillary muscle 34 and in close proximity to the pericardial sac 39.

FIG. 4 is a diagram showing the original principle of the present invention which consists in repositioning the posterior papillary muscle 34. Under radiologic control a guide wire 51 has been directed through the coronary orifice 29 into the posterior interventricular vein 33. Through expansion of a balloon 54 within the posterior interventricular vein 33 the papillary muscle 34 is displaced medially towards the cavity 35 of the left ventricle (arrow 56) because it is retained by the pericardial membrane 39.

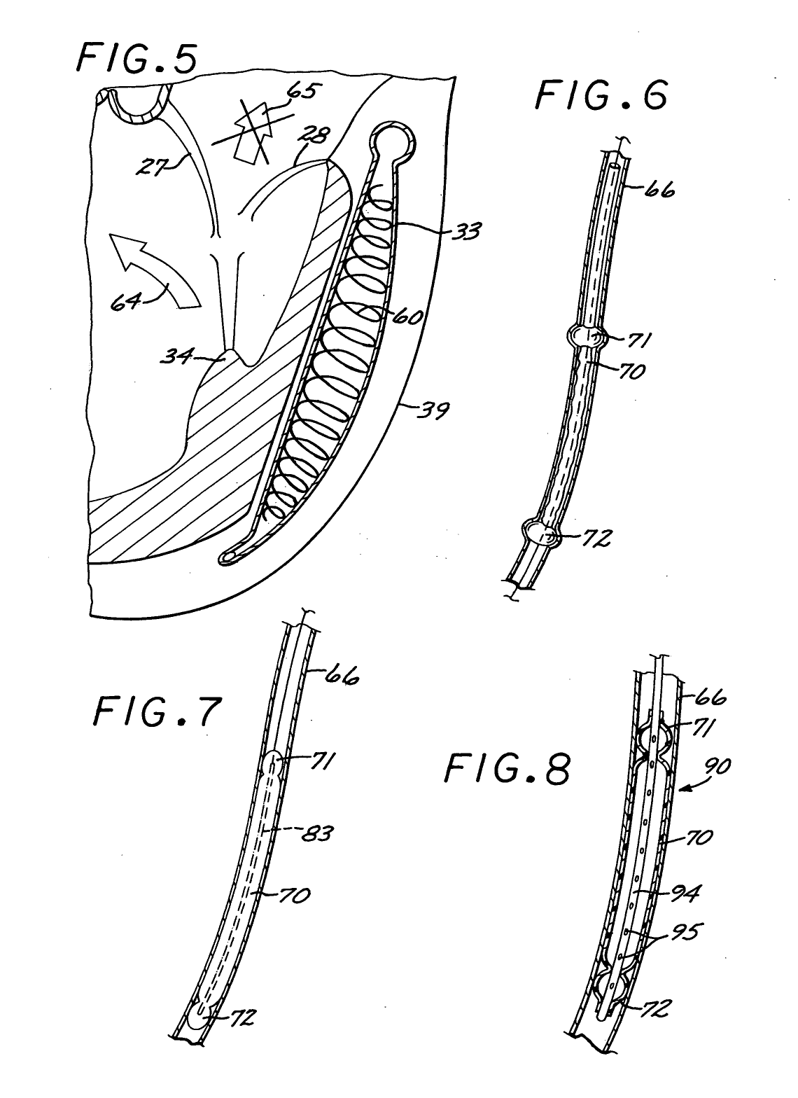

In FIG. 5 a self expandable stent 60 of various embodiments is placed in the posterior interventricular vein 33 that pushes inwards the posterior papillary muscle 34 while avoiding its lateral displacement because of the presence of the pericardial membrane 39. A clear arrow 64 shows medial displacement of the papillary muscle 34 and crossed-out arrow 65 represents the disappearance of the mitral regurgitation.

FIG. 6 shows one of the preferred embodiments of the present invention. To avoid bleeding due to the possible disruption of the posterior interventricular vein by the expansion of a balloon or stent a small bore catheter 66 carries the expandable balloon 70 and proximal 71 and distal 72 small hemostatic balloons. The small balloons 71 and 72 can have radio-opaque markers (not shown) to guide their correct placement within the posterior interventricular vein 33 (not shown in this figure). Once properly located within the vein 33, the hemostatic balloons 71 and 72 are inflated first thereby blocking the blood flow through the vein 33. This is followed by expansion of the central large balloon 70 without the danger of bleeding if the posterior vein 33 were to be torn inadvertently. Occlusion of the posterior interventricular vein 33 has no deleterious effects.

In FIG. 7 the papillary muscle 34 is displaced towards the left ventricular cavity 35 by the displacement of the whole posterior interventricular vein 33. However, to avoid a predominant lateral displacement of the posterior interventricular vein 33 towards the pericardium, a pre-shaped stiff rod 83 is placed centrally within the large balloon 70. In the figure the large balloon 70 and two hemostatic balloons 71 and 72 are shown collapsed within the small bore catheter 66.

In another embodiment shown in FIG. 8, the device 90 in addition to carrying an expandable balloon 70 or stent (not shown) and proximal 71 and distal 72 small balloons as above described, the device 90 also has a central catheter 94 with side holes 95. After the device 90 has been placed into the correct position, both small occluding balloons 71 and 72 are inflated stopping the blood flow in the posterior interventricular vein (not shown in this figure).The balloon 70 is then expanded and a chemical compound that clots the blood or a substance that instantly polymerizes when in contact with blood, is injected through the holes 95 of the catheter 94. An example of this type of substance is Hystoacril that adheres to the vascular endothelium occluding the vascular lumen instantly and permanently (R Villavicencio et al. Selective Coronary Artery Fistula Embolization with Hystoacryl during Percutaneous Coronary Angioplasty. J Invasive Cardiol 2003; vol 15:80-83, incorporated herein by reference).

Another preferred embodiment of the present invention is shown diagrammatically and in principle in FIG. 9. Instead of expanding the posterior interventricular vein 33 with a balloon or a stent, in this embodiment, a pre-shaped stiff rod 102 is used. This rod 102 is placed within the vein 33 attached to a delivery and fixedly positioning guide wire device 101 which is shown, in part in FIGS. 11 and 12. When the rod 102 is properly placed, the vein 33 is displaced medially and consequently the papillary muscle 34 (not shown in this figure) moves medially also. The rod 102 can be rotated as long as it is still attached to the wire insertion and fixating device 101. The instrument of attachment may be a screw, locking device, pin, breakaway, or other standard method of attachment/detachment. The wire insertion device 101 is used to extend the rod 102 to push it into position, rotate the rod 102 to achieve optimum position within the vein 33, and then hold the rod 102 during permanent fixation. While still attached the rod 102 is rotated until it reaches appropriate position. This may be done by fluoroscopy or echocardiogram monitoring. Simultaneously, transthoracic or transesophageal echocardiogram can be used to monitor real time the changes in mitral regurgitation. Radio-opaque markers can be placed at specific points of the rod 102 to help the operator (nor shown). The proper position is that which achieves the least amount of mitral regurgitation. This may involve rotating the rod 102 or changing the rod 102 for another one of different stiffness, degree of eccentricity, length of medial segment, or shape. The rod 102 could be made of metal, plastic, nitinol, stainless steel, or any material with the above properties. Its cross-sectional shape could be that of a wire (cylindrical and thin) or any other shape that can place maximum stress against the left ventricular wall against the posterior papillary muscle while spreading the opposing force against the posterior interventricular vein 33. Also a series of rods 102 may be necessary to be available for the surgeon (not shown) for placement in patients with differing positions of the posterior papillary muscle 34 and/or differing amounts of stiffness necessary to move the muscle 34. After it is fixed in place, the rod 102 is detached from the wire insertion device 101 and the delivery catheter (not shown in FIG. 9) and wire insertion and fixating device 101 (shown in FIGS. 11 and 12) are removed.

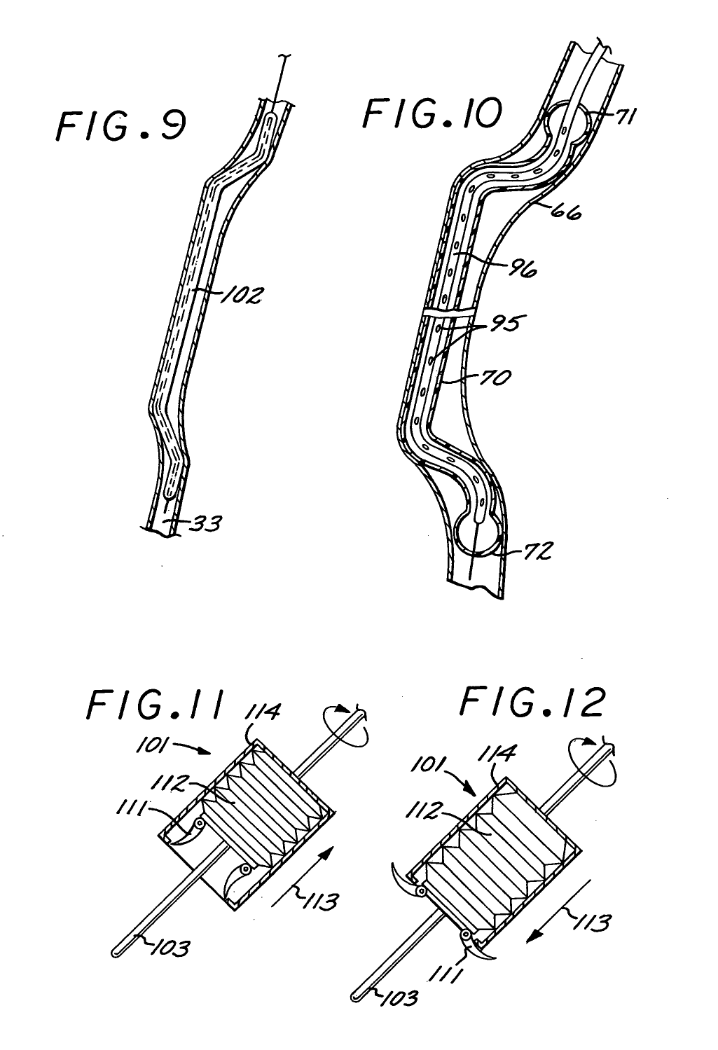

After the rod 102 has been placed into proper position, a method of fixation in the proper position is necessary. This may be accomplished by balloons that are left in place, or by a material that can be inserted to fix the wire or hooks or pressure fixation or glue or springs. An alternative is to inject fast-setting glue through the catheter. This may be done by direct injection of polymers through side holes 95 while stopping blood flow with proximal 71 and distal 72 balloons. FIG. 10 is an example. Although for simplicity of illustration it does not show the rod 102, it shows the vein 66 and a first catheter 70 that carries the balloons 71 and 72 and a second catheter 96 that has side holes 95.

FIGS. 11 and 12 show an exemplary device 101 used in the present invention, designed to maintain in position the pre-shaped stiff rod 102 within the posterior interventricular vein 33 (not shown in these two figures). The central catheter 103 of the device 101 is attached to the stiff rod 102 (not shown in these two figures). The device 101 has hooks 111 that when expanded penetrate through the walls of the vein 33. A threaded torque mechanism 112 moves up or down within a threaded hollowed catheter 114. These up and down movements, shown by the arrows 113 along the rod 102 move inwards or outwards several hooks (111 that penetrate the walls of the vein 33 (not shown in these two figures).

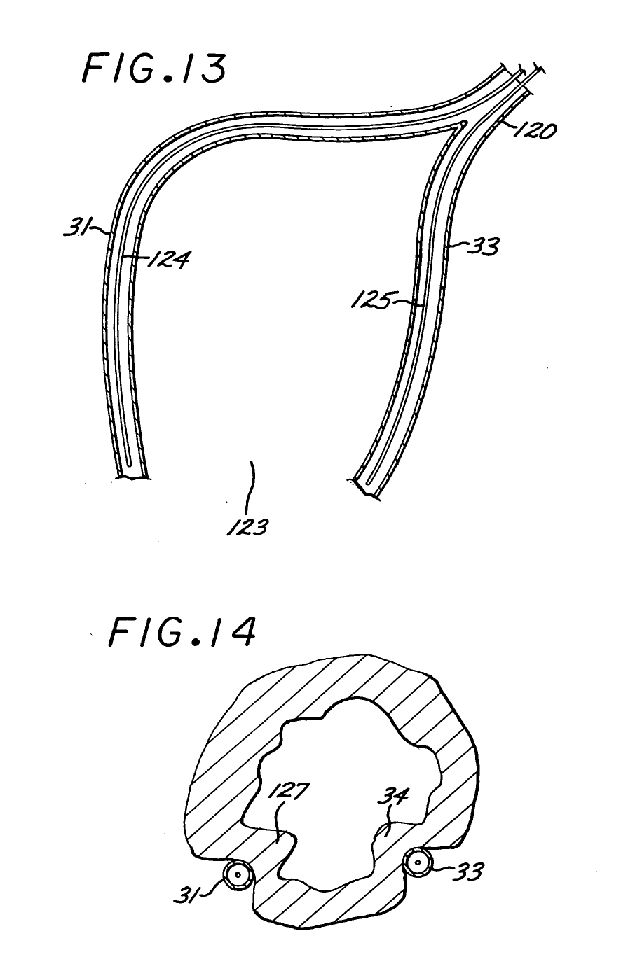

Another preferred embodiment of the present invention, shown in FIGS. 13 and 14, is based on the topographic anatomy of the venous system of the heart. The coronary sinus 120 is mainly formed by the posterior 31 and anterior 33 interventricular veins. They both run from the atrioventricular groove towards the heart's apex 123. FIG. 13 shows two separate guide wires 124 and 125 placed within the anterior 33 and posterior 31 interventricular veins. These guide wires serve for inserting stiff rods (not shown in this figure) or magnets (not shown in this figure).

FIG. 14 is a transverse section of the left ventricle at the level of the posterior 34 and anterior 127 papillary muscles. The posterior 33 and anterior 31 interventricular veins run epicardially towards the ventricular apex and close to the posterior 34 and anterior 127 papillary muscles. Insertion of different types of rods threaded along the guide wires 124 and 125 forces the papillary muscles 34 and 127 towards the left ventricular cavity 123

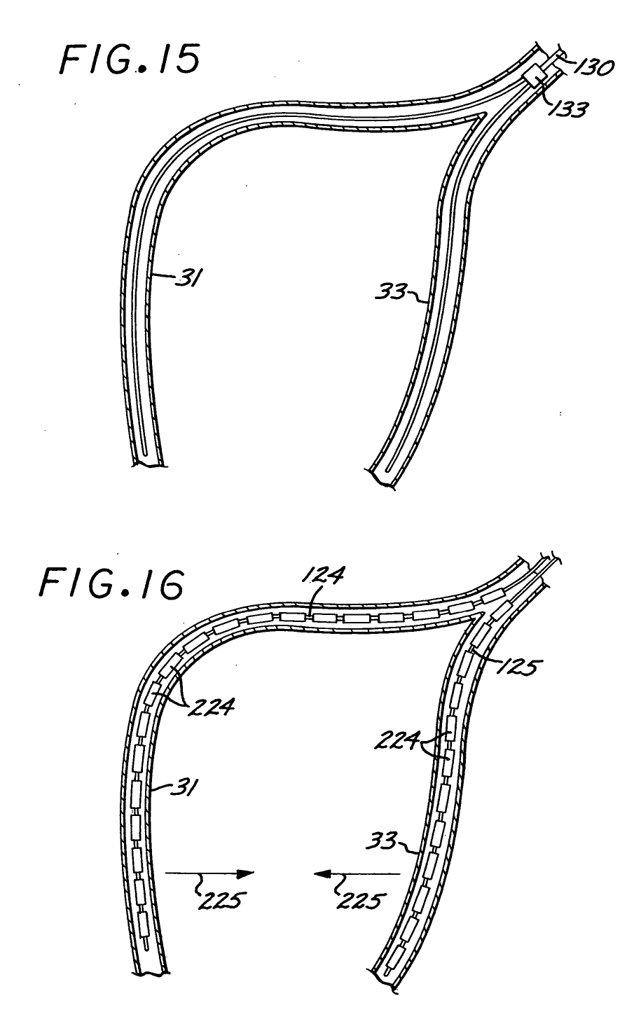

FIG. 15 shows that by placing a stiff rod 130 substantially in the shape of an inverted “U” with its both arms in the anterior 31 and posterior 33 interventricular veins joined to a horizontal member 133 the distance between the veins 31 and 33 can be reduced. FIG. 14 shows how the anterior 127 and posterior 34 papillary muscles are brought closer together by the approximation of the anterior 31 and posterior 33 interventricular veins.

FIG. 16 shows another alternative based on the same principle as above described. Instead of bringing close together the interventricular veins 31 and 33 with an inverted “U” shaped rod, magnets are used. After placing guide wires 124 and 125 into the anterior 31 and posterior 33 interventricular veins, a series of magnets 224 are delivered along the guide wires 124 and 125. After removal of the guide wires the magnets 224 force the veins 31 and 33 closer together and consequently, the papillary muscles also, as shown by the arrows 225.

What is claimed is:

1. A method to reduce the transverse diameter of the heart ventricles of a mammal in need of such reduction for the purpose of ameliorating functional mitral regurgitation, the method comprising:
a step of placing mechanical means configured and dimensioned to fit in at least one of the posterior interventricular and of the posterior interventricular vein, said mechanical means being adapted for pushing the papillary muscle in a direction that reduces the transverse diameter of the heart ventricle.

2. A method in accordance with claim 1 wherein the mechanical means are placed into the vein percutaneously.

3. A method in accordance with claim 1 wherein the mechanical means are placed into the vein endovascularly.

4. A method in accordance with claim 1 wherein the step of placing mechanical means comprises displacing medially the posterior papillary muscle in the heart of a mammal by the mechanical means.

5. A method in accordance with claim 1 wherein the step of placing mechanical means comprises displacing medially the posterior papillary muscle in the heart of a mammal by the mechanical means and said step of displacing comprises the step of placing the mechanical means into both the posterior interventricular vein and into the anterior interventricular vein.

6. An apparatus comprising mechanical means for medially displacing the left ventricular wall of the heart of a mammal in need of such replacement to ameliorate functional mitral regurgitation, the mechanical means being adapted for being placed in at least one of the posterior or anterior interventricular veins of the mammal.

7. The apparatus in accordance with claim 6 wherein the mechanical means are selected from a group consisting of a collapsible and expandable balloon, balloon expanding stent and a self expanding stent, said mechanical means being configured and dimensioned to be placed within at least one of said veins.

8. The apparatus in accordance with claim 6 wherein the mechanical means comprise an eccentrically shaped rigid body configured and dimensioned to be incorporated in at least in one of the posterior and anterior interventricular veins of the heart, said rigid body being capable of being rotated within said vein whereby when rotated selective pressure is applied against the left ventricular wall.

9. An apparatus in accordance with claim 6 wherein the mechanical means comprise a plurality of small magnets and a pair of guide wires, said magnets being threaded on the guide wires, the magnets being configured and dimensioned to fit within the anterior and posterior interventricular veins of the heart whereby when placed into said veins attraction of the magnets brings both veins closer together and consequently brings both papillary muscles closer together.

10. An apparatus in accordance with claim 6 wherein the mechanical means comprise two pre-shaped memory rods configured and dimensioned to fit within the anterior and posterior interventricular veins of the heart and bound together at the level of the coronary sinus to form an inverted U shaped object which when positioned in the anterior and posterior interventricular veins brings closer said veins and consequently brings the papillary muscles closer.

11. An apparatus comprising mechanical means for medially displacing the left ventricular wall of the heart of a mammal in need of such replacement to ameliorate functional mitral regurgitation, the mechanical means being adapted for being placed in at least one of the posterior or anterior interventricular veins of the mammal, said mechanical means including a delivery catheter having the items selected from the group consisting of a collapsible and expandable balloon having small collapsible balloons and a stent having small collapsible balloons placed proximal and distal to the collapsible balloon the balloons in their expanded state serving for blocking the lumen of the posterior or anterior interventricular veins of the heart thereby avoiding bleeding if either the anterior or posterior interventricular veins were to rupture.

12. An apparatus in accordance with claim 11 where the delivery catheter include ports for exuding drugs that induce blood clotting or substances that polymerize when in contact with blood between the expanded small balloons.

13. An apparatus comprising mechanical means including a balloon or a stent and a rigid body configured and dimensioned to be incorporated within the posterior or anterior or both interventricular veins of the heart of a mammal in need of such apparatus for ameliorating functional mitral regurgitation, the mechanical means being adapted for limiting the outward dilatation of the balloon and promoting the inwards dilation towards the left ventricular wall of the heart is promoted.
